(19) United States
(12) Patent Application Publication
(10) Pub. No.: US 2024/0173278 A1
(43) Pub. Date: May 30, 2024

(54) SYSTEMS AND METHODS FOR SUPPLYING NUTRITIONAL SUPPLEMENTS THAT ELIMINATE PERVASIVE ODORS

(71) Applicant: Zinpro Corporation, Eden Prairie, MN (US)

(72) Inventors: Peter A. Stark, Inver Grove Heights, MN (US); Michael Thomas Socha, Rogers, MN (US)

(21) Appl. No.: 18/059,142

(22) Filed: Nov. 28, 2022

(51) Int. Cl. A61K 31/19; A23K 20/105; A23K 50/10

(52) U.S. Cl. CPC A61K 31/19; A23K 20/105; A23K 50/10

(57) ABSTRACT

The present disclosure describes methods for supplying nutrients to ruminants, swine, and poultry. Beneficially, the methods and systems disclosed eliminate pervasive odors characteristic of the nutrients. The methods and systems relate to combining isoacids with liquid feed, blended solid animal feed, or total mixed ration (TMR) and delivering the supplemented feed to animals. Preferably, the isoacids are in a liquid form when combined with a feed.

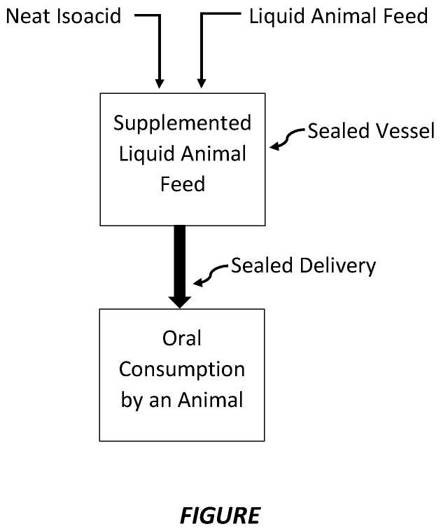

### TECHNICAL FIELD

The present disclosure relates generally to methods for supplying nutrients to ruminants, swine, and poultry that eliminate pervasive odors characteristic of the nutrients. The methods and systems relate to combining isoacids with liquid feed in and delivering the supplemented feed to animals.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Volatile fatty acids such as butyric acid, isobutyric acid, 2-methylbutyric acid, isovaleric acid, and valeric acid are known feed supplements for ruminants, swine, and poultry. For instance, when used as a feed supplement for dairy cattle, milk production is increased. Additionally, these fatty acids have a role as specific nutrients for the ruminal cellulolytic bacteria that act to degrade fibrous feed material during digestion. Further, the fatty acids seem to have a general positive influence on microbial fermentation. However, these volatile acids have a strong odor. The odor is a significant problem to the human workers producing, handling, and/or distributing the acids. Such problems can include nausea and vomiting due to the strong odor.

Previous efforts to reduce the odor include U.S. Pat. No. 4,376,790, which relates to decreasing odor by making ammonium and calcium salts of fatty acids. Additionally, U.S. Pat. No. 10,034,843 relates to overcoming the odor problem by producing feed supplements that are odor free by reacting a pendant carboxylic acid on a water soluble polycarboxylic acid, a polyvalent metal salt, and the odorous volatile fatty acid. The polyvalent metal ion is bonded to the pendant carboxylic acid group of the polycarboxylic acid and the carboxylic acid group of the fatty acid. Another approach has been to putrid odor problems of low molecular weight volatile fatty acids as nutritional supplements for livestock and poultry by reacting a calcium ion source with low molecular weight volatile fatty acid in a solid phase neat reaction, and then using the reaction product as a nutritional supplement. These known solutions require processing of the fatty acids which adds to the cost and time of making and distributing the feed supplements.

There is a continuing need for convenient low-cost methods to lower the odor so as to make volatile fatty acid derived fermentation enhancers a viable feed supplement product.

### BRIEF SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

Described herein is a method for supplying nutritional supplements to a livestock animal for oral consumption comprising combining an isoacid with a liquid to form a supplemented liquid and thereafter delivering the supplemented liquid to an animal for oral consumption. Another embodiment disclosed herein is to mix the liquid to a total mixed ration (TMR) on a farm. Another embodiment disclosed herein is a method of supplying nutritional supplements to a livestock animal by adding the liquid isoacid to an animal feed in a feed mill to form a blended animal feed comprising the isoacids. In an embodiment, the isoacid comprises an isobutyric acid, a 2-methylbutyric acid, an isovaleric acid, a straight-chain valeric acid, butyric acid or combinations thereof. In an embodiment, the liquid comprises a liquid animal feed. In an embodiment, the isoacid is in a liquid form and preferably not in a salt form, and/or does not comprise additional pendant groups. In an embodiment, the isoacid is combined with the liquid in a container and/or the supplemented liquid is delivered to the animal via pipes and/or pumps. In a preferred embodiment, the container is sealed or closed; however, in some embodiments an open container or tank can be used that releases minimal odor based on the size of the container opening. In another embodiment, the isoacids are stored in a container and then processed in a feed mill so that it is blended into animal feed with other feed ingredients to form a blended animal feed comprising the isoacids; the blended animal feed is then delivered to animals.

In an embodiment, the supplemented liquid comprises from about 0.01 wt-% to about 75 wt-% of the isoacid and/or the supplemented liquid comprises the isoacid in an amount sufficient to increase milk production and/or to provide an improved average daily gain (ADG) for the livestock, including, ruminants, swine, and/or poultry. In a more preferred embodiment, the supplemented liquid comprises from about 0.1 wt-% to about 65 wt-% of the isoacid. In a most preferred embodiment, the supplemented liquid comprises from about 1 wt-% to about 50 wt-% of the isoacid.

In an embodiment, the blended animal feed comprises from about 0.01 wt-% to about 75 wt-% of the isoacid and/or the blended animal feed comprises the isoacid in an amount sufficient to increase milk production and/or to provide an improved average daily gain (ADG) for the livestock, including, ruminants, swine, and/or poultry. In a more preferred embodiment, the blended animal feed comprises from about 0.1 wt-% to about 65 wt-% of the isoacid. In a most preferred embodiment, the blended animal feed comprises from about 1 wt-% to about 50 wt-% of the isoacid.

In an embodiment, the method reduces odors associated with the isoacids by at least about 50%, preferably at least by about 60%, more preferably at least by about 70%, even more preferably at least by about 80%, or most preferably at least by about 90%. In a preferred embodiment, the supplemented liquid is stored in a container prior to delivery to the animal; more preferably the container is closed and/or has a small opening; most preferably the container is sealed. In a preferred embodiment, the isoacids are part of a blended animal feed. Beneficially, the blended animal feed can reduce the odor of the isoacids; in a preferred embodiment, the blended animal feed can be stored in a container, more preferably a closed or sealed container.

Described herein is a method of feeding an animal a supplemented animal feed comprising combining an isoacid with a liquid animal feed in a container to form a supplemented animal feed and thereafter delivering the supplemented animal feed to an animal for oral consumption, most preferably the supplemented animal feed remains in a container until delivery to the animal. In an embodiment, the animal is a ruminant, swine or poultry. In an embodiment, the isoacid comprises an isobutyric acid, a butyric acid, a 2-methylbutyric acid, an isovaleric acid, a straight-chain valeric acid, or combinations thereof. In an embodiment, the supplemented liquid comprises from about 0.01 wt-% to about 75 wt-% of the isoacid and/or the supplemented liquid comprises the isoacid in an amount sufficient to increase milk production and/or to provide an improved average daily gain (ADG) for the livestock. In an embodiment, the isoacid is provided in a liquid form, preferably that is not a salt and/or does not comprise additional pendant groups, when it is added to the liquid feed and/or blended animal feed. But other compounds could be added to the liquid. It is within the scope of the disclosure if the isoacid reacts with other feed or ingredients in a feed such that it forms a salt.

Described herein is a method for reducing the odor of nutritional animal supplements comprising combining an isobutyric acid, a 2-methylbutyric acid, an isovaleric acid, a straight-chain valeric acid, butyric acid, or combinations thereof with a liquid animal feed in a container to form a supplemented animal feed, wherein the isobutyric acid, 2-methylbutyric acid, isovaleric acid, straight-chain valeric acid, or butyric acid is in a liquid form and preferably not in a salt form (at the time of mixing with liquid or solid animal feed), and does not comprise additional pendant groups, and thereafter delivering the supplemented animal feed to an animal, wherein the supplemented animal feed remains until (mixed with traditional liquid animal feed) delivery to the animal. In an embodiment, the method reduces odors associated with the isoacids by at least about 50%, or at least about 90%.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE FIGURE

Several embodiments in which the present disclosure can be practiced are illustrated and described in detail, wherein like reference characters represent like components throughout the detailed description. The FIGURE is provided for exemplary purposes and does not include all steps or equipment that may be employed practicing the systems and methods disclosed herein.

FIGURE shows a flow chart of the method wherein neat isoacids are added to a liquid animal feed in a container, and then distributed to an animal in an environment.

An artisan of ordinary skill in the art need not view, within isolated FIGURE, the near infinite number of distinct permutations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

The present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

Numeric ranges recited within the specification are inclusive of the numbers within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numerical values within that range, for example, 1, 2, 3, 4, 5, and 6, and decimals and fractions, for example, 1.2, 3.8, 1½, and 4¾ This applies regardless of the breadth of the range.

The term “about,” as used herein, refers to variations in size, distance or any other types of measurements that can be resulted from inherent heterogeneous nature of the measured objects and imprecise nature of the measurements itself, including, but not limited to, temperature, mass, volume, weight, density, hardness, strength, thickness, ratios, concentration, odor, and percentages. The term “about” also encompasses variation in the numerical quantity that can occur, for example, through typical measuring or handling procedures in the real world; through inadvertent error in these procedures; through differences in the manufacture, source, or purity of the ingredients used to make the device or carry out the methods, and the like. Whether or not modified by the term “about”, the claims include equivalents to the quantities.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The term “container” as used herein, includes, but is not limited to, a vessel, bucket, tote, tank, drum, or bag. The container can be open; however, if open, it preferably has a small opening so as to reduce the amount of noxious odor. The container can include a vent, an opening configured for pouring, a pump, and/or a mechanism for fluid communication. In a preferred embodiment, the container is closed (such can include a container with a closeable opening). In a most preferred embodiment, the container is sealed. As used herein, “sealed” is synonymous with airtight, wherein gases cannot substantially escape, enter, or pass-through. For instance, a sealed container refers to a container wherein gases cannot substantially escape, enter or pass-through the container. Except when being opened for tubing, connections etc. No significant exposure is possible.

The term “livestock” as used herein, includes, but is not limited to, ruminants, swine, and poultry. Ruminants includes, but is not limited to, cattle, goats, and sheep.

The term, “supplemented feed” as used herein refers to a liquid feed, dry blended animal feed, and/or a TMR which has been supplemented with the isoacid.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The disclosure herein is related to a method and/or system for reducing the odor of volatile fatty acids, also known as isoacids, as a nutritional supplement for an animal. An example of the method is outlined in the FIGURE. As shown in the FIGURE, the method comprises obtaining an isoacid, combining the isoacid with a liquid, preferably in a closed or scaled environment, to form a supplemented liquid, and storing and/or delivering the supplemented liquid to an animal via a pumping system or for oral consumption by the animal.

A further embodiment comprises obtaining an isoacid, combining the isoacid with a solid animal feed in a feed mill to form a blended animal feed, which comprises the isoacid, and delivering the blended animal feed to the animals for consumption. Any suitable solid animal feeds can be combined with the isoacid. Preferred solid animal feeds include, but are not limited to, grains, oilseeds, oilseed meals, forage byproducts, oilseed byproducts animal-derived products (including, but not limited to, chicken, beef, fish), vegetables, nuts, seeds, fruits, tubers, or a mixture thereof.

Still a further embodiment comprises mixing the isoacid and/or liquid feed with a total mixed ration (TMR) on a farm and delivering the TMR to the livestock.

The isoacid can be added to a lick tank (i.e., tank which contains feed comprising the isoacid) such that a wheel or other lick apparatus, contacts the feed comprising the isoacid, which is then delivered to the animal for consumption.

As used herein, an “isoacid” refers to the volatile fatty acids isobutyric acid, 2-methylbutyric acid and isovaleric acid, the straight-chain volatile fatty acid valeric acid, and butyric acid.

In an embodiment, the isoacid comprises isobutyric acid, 2-methylbutyric acid, isovaleric acid, valeric acid, butyric acid, and combinations thereof. In an embodiment, the isoacid is one of more of isobutyric acid, 2-methylbutyric acid, butyric acid, isovaleric acid, and valeric acid in a liquid form. In an embodiment, the isoacid is in the original acid form (referred to as “neat”), without processing into a salt and/or without additional pendant groups. In an embodiment, the isoacid is reacted to form an ester, amide, or a salt. In an embodiment, the isoacid has not been processed into another compound to reduce the odor of the isoacid. In an embodiment, the isoacid has not been processed in any manner to reduce the odor of the isoacid prior to the methods described herein. In an embodiment, the isoacid is diluted with water or another liquid diluent. In an embodiment, the isoacid is not diluted. It should be understood that the isoacid, once mixed with other feed components (liquid or solid) can interact and form a salt and/or solid.

In an embodiment, the liquid isoacid is combined with a liquid animal feed. The combination can be referred to as a supplemented animal feed. As used herein, a liquid animal feed comprises any liquid animal feed as known in the art. For instance, a liquid animal feed may comprise various raw materials of agricultural, plant, animal, marine, or chemical origin and liquid additives such as molasses, vegetable oils, animal fats, milk-whey products, sugars, protein concentrates, nutrients, fiber and the like. In an embodiment, the liquid comprises water. In an embodiment, the liquid is a liquid animal feed. In an embodiment, the liquid is water or another edible liquid.

In an embodiment, the liquid is contained within a container. In an embodiment, the isoacid and the liquid are combined in a container.

In an embodiment, the isoacid and liquid are combined in a container. In an embodiment the supplemented liquid remains until delivery to an animal. In an embodiment, the supplemented liquid is stored before delivery to an animal. In an embodiment, the supplemented liquid is stored in a container. In an embodiment, the supplemented liquid is delivered to the animal in a container. In an embodiment, the supplemented liquid is delivered and/or transported via pipes and/or pumps, or any sealed transportation and/or delivery system as known in the art.

In an embodiment, the supplemented liquid comprises from about 0.01 wt-% to about 75 wt-% of an isoacid. In an embodiment, the supplemented liquid comprises from about 0.1 wt-% to about 65 wt-% of an isoacid. In an embodiment, the supplemented liquid comprises from about 0.5 wt-% to about 50 wt-% of an isoacid. In an embodiment, the supplemented liquid comprises from about 1 wt-% to about 35 wt-% of an isoacid.

In an embodiment, the blended animal feed comprises from about 0.01 wt-% to about 55 wt-% of an isoacid. In an embodiment, the blended animal feed comprises from about 0.1 wt-% to about 45 wt-% of an isoacid. In an embodiment, the blended animal feed comprises from about 0.5 wt-% to about 35 wt-% of an isoacid. In an embodiment, the supplemented liquid comprises from about 1 wt-% to about 30 wt-% of an isoacid.

In an embodiment, the animal is a ruminant, swine or poultry. In an embodiment, the animal is a ruminant. As used herein, a ruminant refers to mammals that chew the cud regurgitated from its rumen. Ruminants comprise cattle, sheep, antelopes, deer, giraffes, and their relatives.

In an embodiment, the supplemented liquid comprises the isoacid in an amount sufficient to increase milk production when fed to a ruminant.

In an embodiment, the blended animal feed comprises the isoacid in an amount sufficient to increase milk production when fed to a ruminant.

In an embodiment, the supplemented liquid comprises the isoacid in an amount sufficient to increase ADG in a livestock animal.

In an embodiment, the blended animal feed comprises the isoacid in an amount sufficient to increase ADG in a livestock animal.

In an embodiment, the supplemented liquid comprises the isoacid in an amount that provides an improved feed efficiency and feed conversion.

In an embodiment, the blended animal feed comprises the isoacid in an amount that provides an improved feed efficiency and feed conversion.

In an embodiment, the methods and systems described herein, reduce the odor associated with isoacids by about 10%, by about 20%, by about 30%, by about 40%, by about 50%, by about 60%, by about 70%, by about 80%, by about 90%, by about 95%, by about 99%, by about 100%. In an embodiment, the methods and systems described herein, reduce the odor associated with isoacids by at least about 10%, by at least about 50%, by at least about 90%.

In an embodiment, the containers, pipes, tubes and other apparatuses which carry the isoacids, liquid feed, or blended animal feed can be cleaned, rinsed, and/or flushed. This can aid in reducing the odor in the container.

Odor can be assessed by any method known in the art. One such method includes the following protocol:

The Odor Assessor: a) must be free of colds or physical conditions that may affect the sense of smell; b) must not chew gum or eat at least 30 minutes prior to the test; c) must refrain from eating spicy foods prior to the test; and d) must not wear perfume cologne or after shave the day of the test.

During an odor test, the odor assessor sniffs a sample of the product as the container is opened approximately six inches directly below the assessor's nostrils. The odor intensity is then qualitatively compared to control samples: isobutyric acid (IBA), 1:10 IBA/water, 1:100 IBA/water, and pure water. A score of zero odor units indicates no smell. A score of three odor units indicates an odor intensity equivalent to 1:100 IBA/water. A score of six odor units indicates an odor intensity equivalent to 1:10 IBA/water. A score of ten odor units indicates an odor as intense as undiluted isobutyric acid. The test is repeated as necessary with the assessor revisiting the controls and test product as often as necessary prior to a qualitative confidence being reached. The assessor then repeats this test on a series of no greater than ten individual test products in one 24-hour period. The individual estimated intensities for three to five assessments and are averaged to the nearest whole number to determine the reportable odor intensity. There will be no odor until the final feed is produced. We have evaluated in a molasses-based feed and the odor is indistinguishable from the blank molasses. In whey-based feed it is noticeable but nothing compared to the pure isoacids.

The present disclosure is also related to a method of feeding an animal a supplemented liquid feed. The method of feeding an animal comprises the method and/or system for reducing the odor of isoacids as described herein.

The present disclosure is also related to a method of supplying nutritional supplements to an animal for oral consumption. The method of supplying nutritional supplements comprises the method and/or system for reducing the odor of isoacids as described herein.

The present disclosure is further defined by the following numbered embodiments:
1. A method for supplying nutritional supplements to an animal for oral consumption comprising:
a) combining a liquid isoacid with an animal feed to form a supplemented feed; and
b) delivering the supplemented feed to an animal for oral consumption.
2. The method of embodiment 1, wherein the isoacid comprises an isobutyric acid, a 2-methylbutyric acid, an isovaleric acid, a straight-chain valeric acid, butyric acid, or combinations thereof.
3. The method of any one of embodiments 1-2, wherein the animal feed comprises a liquid animal feed.
4. The method of any one of embodiments 1-3, wherein the animal feed comprises a solid animal feed and wherein the supplemented feed is a blended animal feed.
5. The method of any one of embodiments 1-2 or 4, wherein the animal feed comprises a TMR.
6. The method of any one of embodiments 1-5, wherein the isoacid is not in a salt form and does not comprise additional pendant groups when combined with the animal feed.
7. The method of any one of embodiments 1-6, wherein the isoacid is combined with the animal feed in a sealed container and/or is delivered to the animal via a lick tank, pipes and/or pumps.
8. The method of any one of embodiments 1-7, wherein the supplemented feed comprises from about 0.01 wt-% to about 75 wt-% of the isoacid.
9. The method of any one of embodiments 1-8, wherein the supplemented feed comprises the isoacid in an amount sufficient to increase milk production in a ruminant.
10. The method of any one of embodiments 1-9, wherein the method reduces odors associated with the isoacids by at least about 50%, or by at least about 90%.
11. The method of any one of embodiments 1-10, wherein the supplemented feed is stored in a container prior to delivery to the animal.
12. The method of embodiment 11, wherein the container is a closed or sealed container.
13. A method of feeding an animal a supplemented animal feed comprising:
a) combining a liquid isoacid with a liquid animal feed in a container to form a supplemented feed; and
b) delivering the supplemented feed to an animal for oral consumption, wherein the supplemented feed remains sealed until delivery to the animal.
14. The method of embodiment 13, wherein the isoacid comprises an isobutyric acid, a 2-methylbutyric acid, an isovaleric acid, a straight-chain valeric acid, butyric acid, or combinations thereof.
15. The method of embodiment 13 or 14, wherein the animal is a ruminant, swine or poultry.
16. The method of embodiment 15, wherein the animal is a ruminant.
17. The method of embodiment 16, wherein the supplemented feed comprises the isoacid in an amount sufficient to increase milk production and/or the ADG when fed to the ruminant.
18. The method of any one of embodiments 13-17, wherein the supplemented liquid comprises from about 0.01 wt-% to about 50 wt-% of the isoacid.
19. The method of any one of embodiments 13-18, wherein the isoacid is not in a salt form and does not comprise additional pendant groups when combined with the liquid animal feed.
20. A method for reducing the odor of nutritional animal supplements comprising:
a) combining a liquid isoacid, wherein the liquid isoacid comprises isobutyric acid, a 2-methylbutyric acid, an isovaleric acid, a straight-chain valeric acid, a butyric acid, or combinations thereof, with an animal feed in a container to form a supplemented feed, wherein at the time of combination the isoacid is not in a salt form and does not comprise additional pendant groups; and
b) delivering the supplemented feed to an animal.
21. The method of embodiment 20, wherein the method reduces odors associated with the isoacids by at least about 50%.
22. The method of embodiment 21, wherein the method reduces odors associated with the isoacids by at least about 90%.
23. The method of any one of embodiments 20-22, wherein the isoacid is combined with the animal feed in a container and/or is delivered to the animal via a lick tank, pipes and/or pumps.
24. The method of embodiment 23, wherein the container is a sealed container.
25. The method of any one of embodiments 20-24, wherein the animal is a ruminant, swine or poultry.
26. The method of embodiment 25, wherein the animal is a ruminant.
27. The method of embodiment 26, wherein the supplemented feed comprises the isoacid in an amount sufficient to increase milk production and/or the ADG when fed to the ruminant.

The above disclosure is intended to be illustrative and not exhaustive. This description will suggest many variations and alternatives to one of ordinary skill in this art. All these alternatives and variations are intended to be included within the scope of the following claims where the term “comprising” means “including, but not limited to”. Those familiar with the art may recognize other equivalents to the specific embodiments described herein which equivalents are also intended to be encompassed by the following claims.

What is claimed is:

1. A method for supplying nutritional supplements to an animal for oral consumption comprising:
a) combining a liquid isoacid with an animal feed to form a supplemented feed; and
b) delivering the supplemented feed to an animal for oral consumption.

2. The method of claim 1, wherein the isoacid comprises an isobutyric acid, a 2-methylbutyric acid, an isovaleric acid, a straight-chain valeric acid, butyric acid, or combinations thereof.

3. The method of claim 1, wherein the animal feed comprises a liquid animal feed.

4. The method of claim 1, wherein the animal feed comprises a solid animal feed and wherein the supplemented feed is a blended animal feed.

5. The method of claim 1, wherein the animal feed comprises a TMR.

6. The method of claim 2, wherein the isoacid is not in a salt form and does not comprise additional pendant groups when combined with the animal feed.

7. The method of claim 1, wherein the isoacid is combined with the animal feed in a sealed container and/or is delivered to the animal via a lick tank, pipes and/or pumps.

8. The method of claim 1, wherein the supplemented feed comprises from about 0.01 wt-% to about 75 wt-% of the isoacid.

9. The method of claim 1, wherein the supplemented feed comprises the isoacid in an amount sufficient to increase milk production in a ruminant.

10. The method of claim 1, wherein the method reduces odors associated with the isoacids by at least about 50%.

11. The method of claim 1, wherein the supplemented feed is stored in a container prior to delivery to the animal.

12. The method of claim 11, wherein the container is a closed or sealed container.

13. A method of feeding an animal a supplemented animal feed comprising:
a) combining a liquid isoacid with a liquid animal feed in a container to form a supplemented feed; and
b) delivering the supplemented feed to an animal for oral consumption, wherein the supplemented feed remains sealed until delivery to the animal.

14. The method of claim 13, wherein the isoacid comprises an isobutyric acid, a 2-methylbutyric acid, an isovaleric acid, a straight-chain valeric acid, butyric acid, or combinations thereof.

15. The method of claim 13, wherein the animal is a ruminant, swine or poultry.

16. The method of claim 13, wherein the animal is a ruminant.

17. The method of claim 16, wherein the supplemented feed comprises the isoacid in an amount sufficient to increase milk production and/or the ADG when fed to the ruminant.

18. The method of claim 13, wherein the supplemented liquid comprises from about 0.01 wt-% to about 50 wt-% of the isoacid.

19. The method of claim 13, wherein the isoacid is not in a salt form and does not comprise additional pendant groups when combined with the liquid animal feed.

20. A method for reducing the odor of nutritional animal supplements comprising:
a) combining a liquid isoacid, wherein the liquid isoacid comprises isobutyric acid, a 2-methylbutyric acid, an isovaleric acid, a straight-chain valeric acid, a butyric acid, or combinations thereof, with an animal feed in a container to form a supplemented feed, wherein at the time of combination the isoacid is not in a salt form and does not comprise additional pendant groups; and
b) delivering the supplemented feed to an animal.
